(19) United States
(12) Patent Application Publication
(10) Pub. No.: US 2009/0082627 A1
(43) Pub. Date: Mar. 26, 2009

(54) MEDICAL APPARATUS

(75) Inventors: Hitoshi KARASAWA, Tokyo (JP); Daisuke ASADA, Tokyo (JP); Keiji HANDA, Tokyo (JP); Sho NAKAJIMA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/233,233

(22) Filed: Sep. 18, 2008

(30) Foreign Application Priority Data
Sep. 20, 2007 (JP) 2007-244205

(51) Int. Cl. A61B 1/045

(52) U.S. Cl. 600/118

(57) ABSTRACT

A medical apparatus includes: a medical device equipped with a driven attitude controller and introduced into a body cavity; a fastener detachably installed on the medical device and used for fastening to a body wall in the body cavity; and an attitude control apparatus equipped with an attitude controller which moves the medical device with respect to the fastener.

### CROSS REFERENCE TO RELATED APPLICATION

This application claims benefit of Japanese Patent Application No. 2007-244205 filed on Sep. 20, 2007, the contents of which are incorporated by this reference.

### BACKGROUND OF THE INVENTION

1. Field of the Invention

The present invention relates to a medical apparatus equipped with a medical device which is fastened in a body, the medical apparatus being capable, in particular, of moving orientation of the medical device from outside the body.

2. Description of the Related Art

As is well known, an endoscope which is a medical device is equipped with an image pickup apparatus, introduced into a body cavity of a patient, and used for various inspections and treatments of an affected area in the body based on observation images shot by the image pickup apparatus.

Such an endoscope is introduced into luminal tracts, i.e., digestive organs such as an esophagus, stomach, large intestine, and duodenum through an anus or oral cavity, or introduced into an abdominal cavity by puncturing a body wall in an area around the navel. Generally, the endoscope has a slender insertion portion, which is inserted into a digestive tract or abdominal cavity.

Recently, to alleviate patients' pain caused by insertion of the insertion portion, capsule-type medical apparatus such as those described in Japanese Patent Application Laid-Open Publication Nos. 2007-14634 and 2007-89893 have been proposed.

Japanese Patent Application Laid-Open Publication No. 2007-14634 discloses a technique for leaving a capsule-type endoscope in a body cavity using two clips which locks the capsule-type endoscope onto tissue in the body cavity. Specifically, with the technique disclosed in Japanese Patent Application Laid-Open Publication No. 2007-14634, to lock the capsule-type endoscope onto the tissue in the body cavity with the two clips, after fastening one of the clips to the tissue in the body cavity, a surgeon fastens the other clip to the tissue in the body cavity by adjusting the clip using a clipping treatment instrument while monitoring images from the capsule-type endoscope.

On the other hand, Japanese Patent Application Laid-Open Publication No. 2007-89893 discloses a technique for leaving a capsule-type endoscope in a body cavity by passing a clip through a hole in a joint coupled to the capsule-type endoscope and fastening the clip to tissue in the body cavity.

### SUMMARY OF THE INVENTION

The present invention provides a medical apparatus comprising: a medical device equipped with a driven attitude controller and introduced into a body cavity; a fastener detachably installed on the medical device and used for fastening to a body wall in the body cavity; and an attitude control apparatus equipped with an attitude controller which moves the medical device with respect to the fastener.

The above and other objects, features and advantages of the invention will become more clearly understood from the following description referring to the accompanying drawings.

### BRIEF DESCRIPTION OF THE DRAWINGS

FIG. 1 is a diagram showing a configuration of an endoscope system which is a medical apparatus according to an embodiment of the present invention;

FIG. 2 is a sectional view showing a configuration of an extracorporeal apparatus according to the embodiment of the present invention;

FIG. 3 is a top view showing a configuration of the extracorporeal apparatus according to the embodiment of the present invention;

FIG. 4 is a sectional view showing a configuration of an intra-abdominal camera according to the embodiment of the present invention;

FIG. 5 is a sectional view taken along line V-V in FIG. 4, according to the embodiment of the present invention;

FIG. 6 is a sectional view illustrating how an abdominal wall fastener is attached and detached to/from a camera body of the intra-abdominal camera according to the embodiment of the present invention;

FIG. 7 is a perspective view illustrating how the abdominal wall fastener is attached and detached to/from the camera body of the intra-abdominal camera shown in FIG. 6, according to the embodiment of the present invention;

FIG. 8 is an overall configuration diagram showing a state in which the intra-abdominal camera is fastened to the abdominal wall, according to the embodiment of the present invention;

FIG. 9 is a diagram showing a state in which the extracorporeal apparatus is installed on the abdomen and the intra-abdominal camera is fastened to the abdominal wall, according to the embodiment of the present invention;

FIG. 10 is a sectional view of the extracorporeal apparatus and intra-abdominal camera in the state shown in FIG. 9, according to the embodiment of the present invention;

FIG. 11 is a sectional view illustrating how the intra-abdominal camera rotates around an axis when the extracorporeal apparatus is manipulated, according to the embodiment of the present invention;

FIG. 12 is a sectional view illustrating how the intra-abdominal camera rotates at an angle with respect to the axis when the extracorporeal apparatus is manipulated, according to the embodiment of the present invention;

FIG. 13 is a sectional view illustrating how a camera body of an intra-abdominal camera and a ball joint connected in a row with an abdominal wall fastener are attached and detached to/from each other, according to a first variation of the embodiment of the present invention;

FIG. 14 is a perspective view illustrating how the camera body of the intra-abdominal camera and the ball joint connected in a row with the abdominal wall fastener in FIG. 13 are attached and detached to/from each other, according to the first variation of the embodiment of the present invention;

FIG. 15 is a sectional view illustrating how an image pickup unit of an intra-abdominal camera and an intracorporeal attitude adjuster connected in a row with an abdominal wall fastener are attached and detached to/from each other, according to a second variation of the embodiment of the present invention;

FIG. 16 is a perspective view illustrating how the image pickup unit of the intra-abdominal camera and the intracorporeal attitude adjuster connected in a row with the abdominal wall fastener in FIG. 15 are attached and detached to/from each other, according to the second variation of the embodiment of the present invention;

FIG. 17 is a perspective view showing a configuration of a coil spring in the intracorporeal attitude adjuster of the intra-abdominal camera, according to the second variation of the embodiment of the present invention; and

FIG. 18 is a perspective view showing a configuration of a self-aligning bearing in the intracorporeal attitude adjuster of the intra-abdominal camera, according to the second variation of the embodiment of the present invention.

### DETAILED DESCRIPTION OF THE PREFERRED EMBODIMENTS

An embodiment of the present invention will be described below with reference to the drawings. The description will be provided by taking a medical apparatus used for laparoscopic surgery as an example.

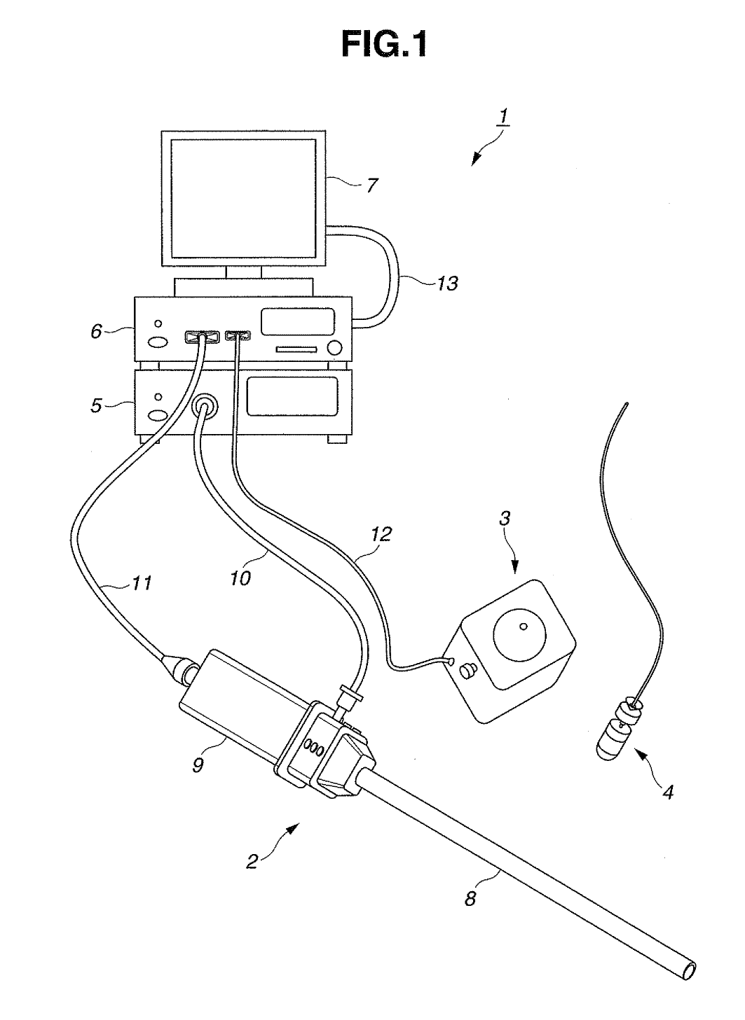

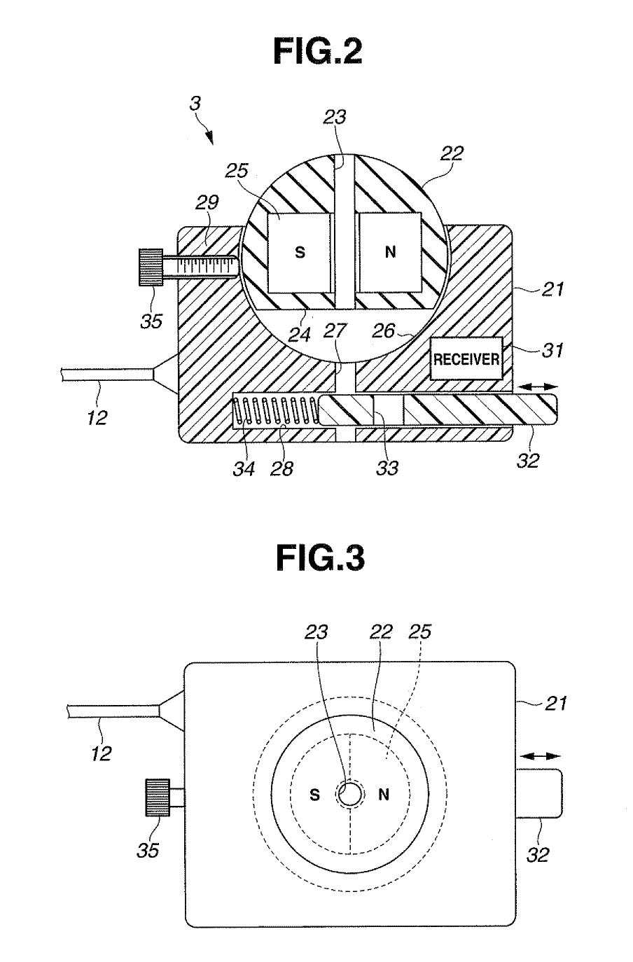

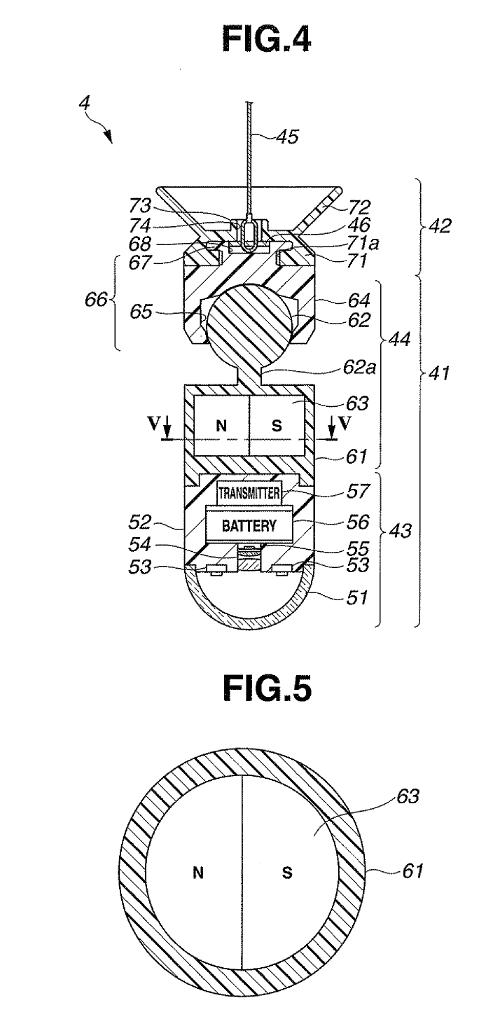

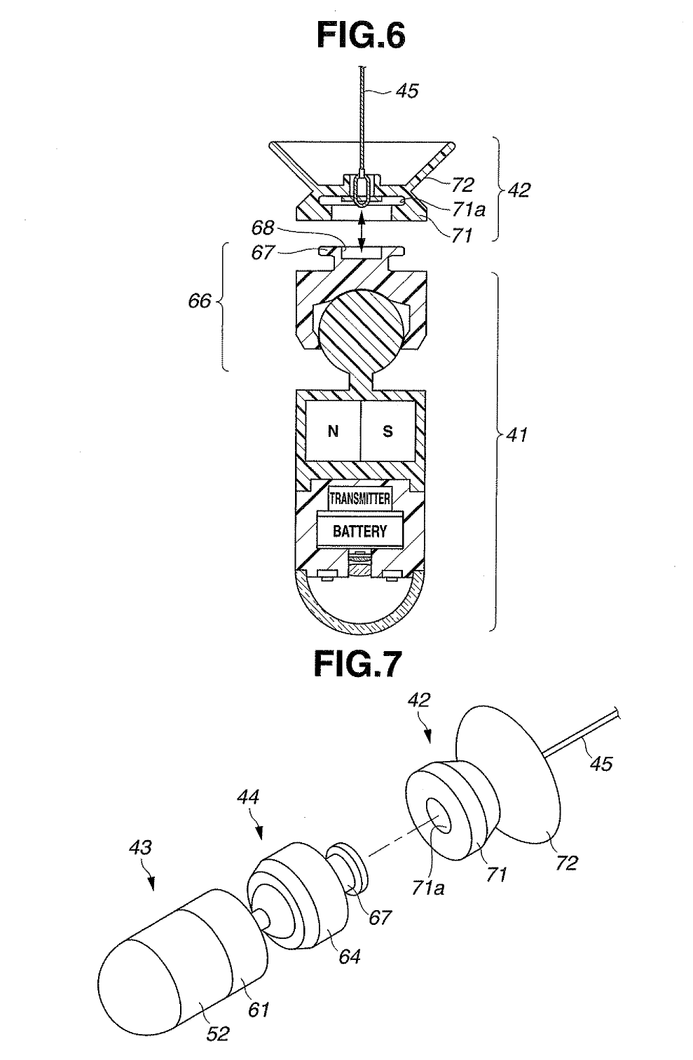

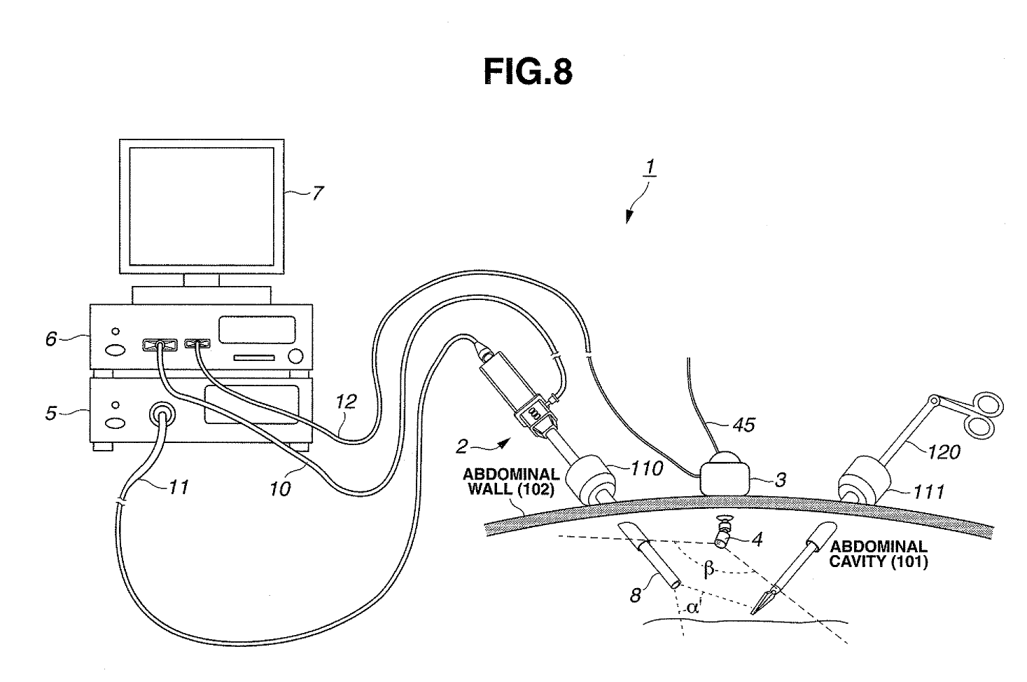

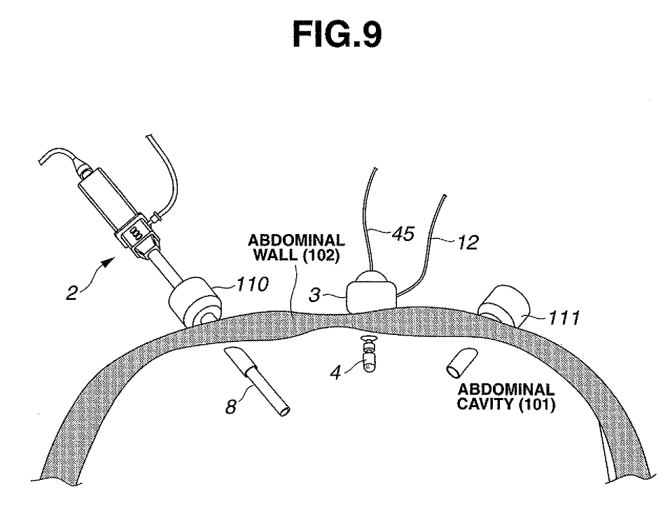

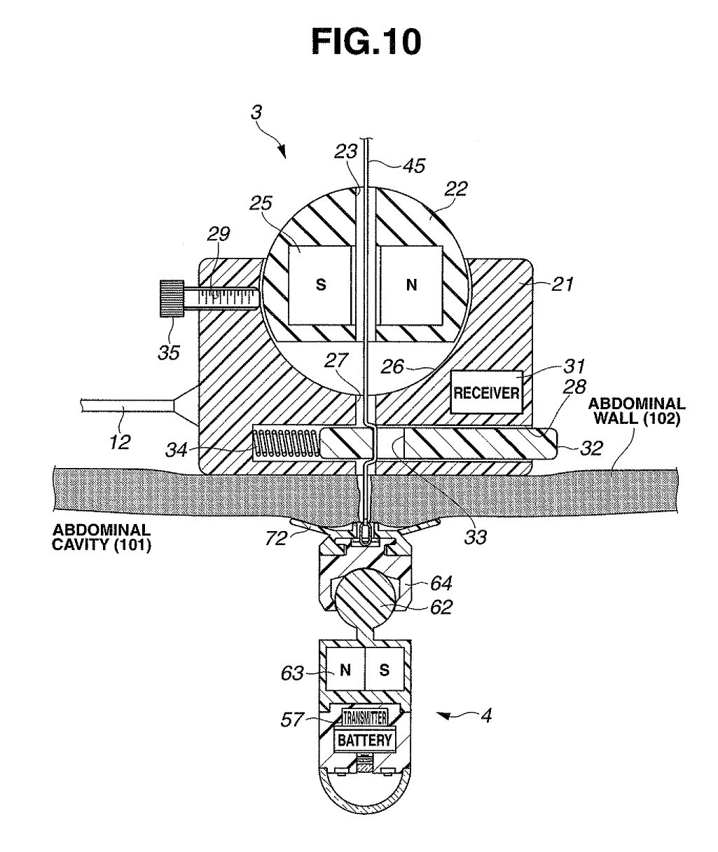

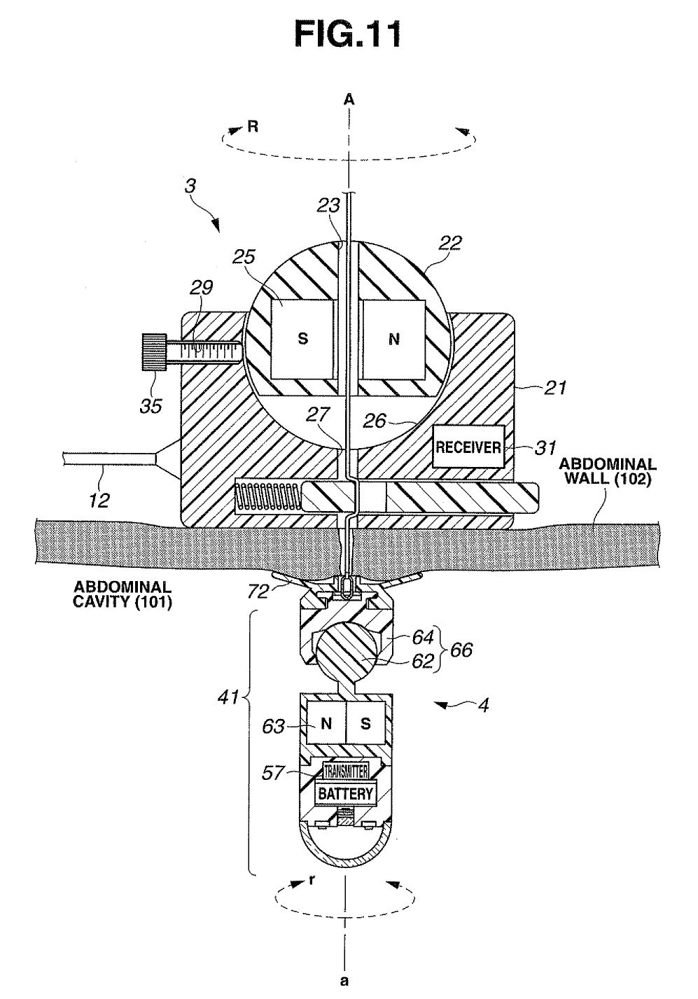

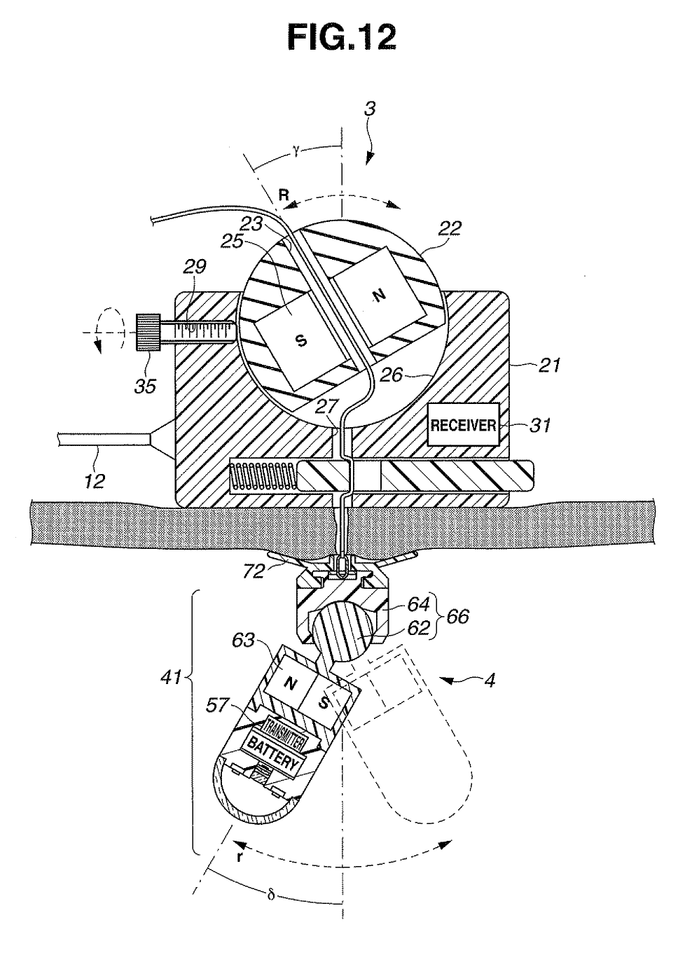

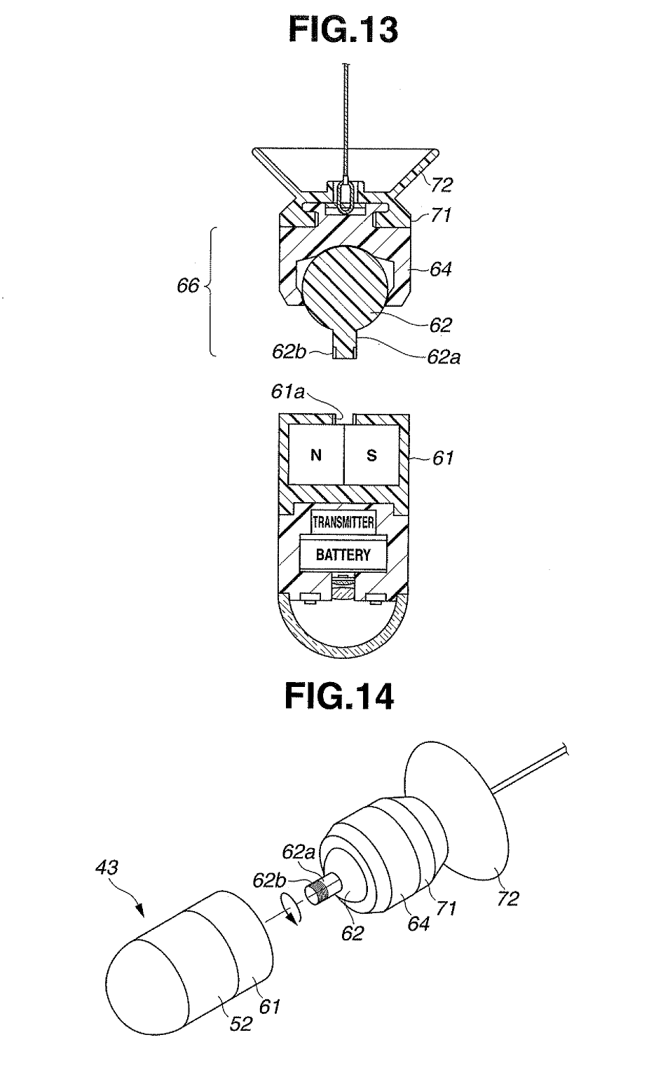

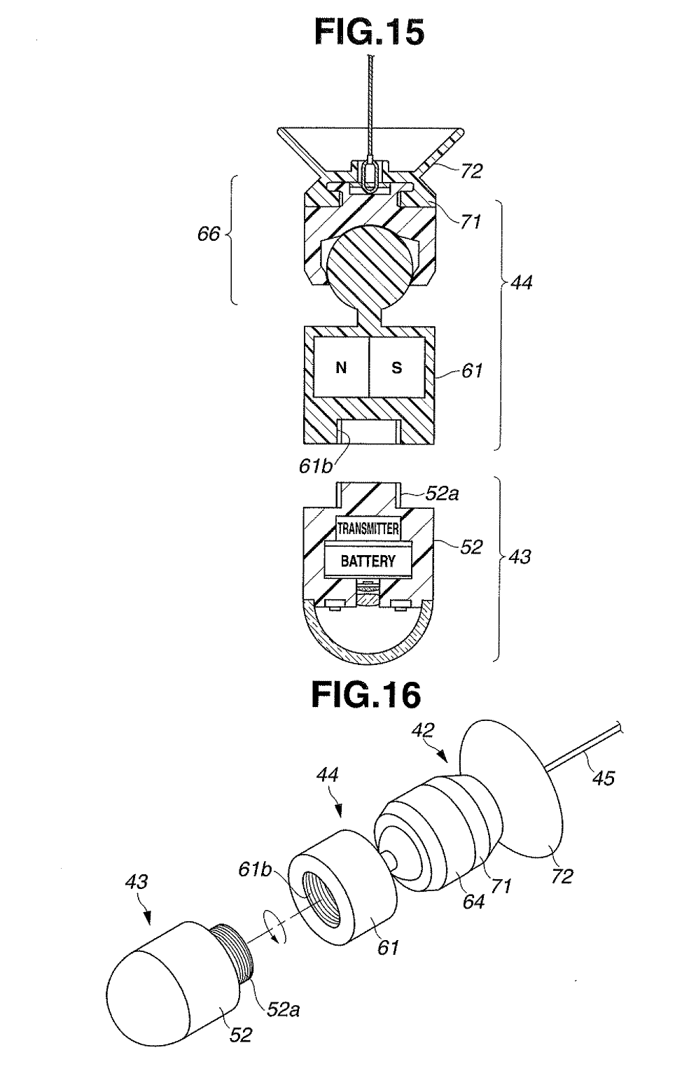

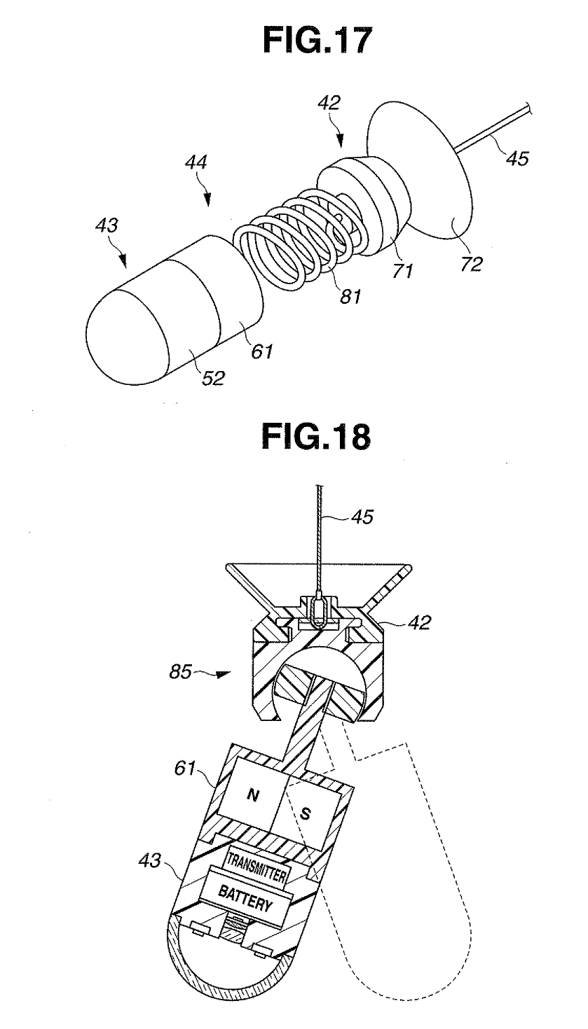

An endoscope system which is the medical apparatus according to the present invention used for laparoscopic surgery will be described below with reference to FIGS. 1 to 18. FIGS. 1 to 18 relate to the embodiment of the present invention, where FIG. 1 is a diagram showing a configuration of an endoscope system which is a medical apparatus; FIG. 2 is a sectional view showing a configuration of an extracorporeal apparatus; FIG. 3 is a top view showing a configuration of the extracorporeal apparatus; FIG. 4 is a sectional view showing a configuration of an intra-abdominal camera; FIG. 5 is a sectional view taken along line V-V in FIG. 4; FIG. 6 is a sectional view illustrating how an abdominal wall fastener is attached and detached to/from a camera body of the intra-abdominal camera; FIG. 7 is a perspective view illustrating how the abdominal wall fastener is attached and detached to/from the camera body of the intra-abdominal camera shown in FIG. 6; FIG. 8 is an overall configuration diagram of the endoscope system showing a state in which the intra-abdominal camera is fastened to the abdominal wall; FIG. 9 is a diagram showing a state in which the extracorporeal apparatus is installed on the abdomen and the intra-abdominal camera is fastened to the abdominal wall; FIG. 10 is a sectional view of the extracorporeal apparatus and intra-abdominal camera in the state shown in FIG. 9; FIG. 11 is a sectional view illustrating how the intra-abdominal camera rotates around an axis when the extracorporeal apparatus is manipulated; FIG. 12 is a sectional view illustrating how the intra-abdominal camera rotates at an angle with respect to the axis when the extracorporeal apparatus is manipulated; FIG. 13 is a sectional view illustrating how a camera body of an intra-abdominal camera and a ball joint connected in a row with an abdominal wall fastener are attached and detached to/from each other, according to a first variation; FIG. 14 is a perspective view illustrating how the camera body of the intra-abdominal camera and the ball joint connected in a row with the abdominal wall fastener in FIG. 13 are attached and detached to/from each other; FIG. 15 is a sectional view illustrating how an image pickup unit of an intra-abdominal camera and an intracorporeal attitude adjuster connected in a row with an abdominal wall fastener are attached and detached to/from each other, according to a second variation; FIG. 16 is a perspective view illustrating how the image pickup unit of the intra-abdominal camera and the intracorporeal attitude adjuster connected in a row with the abdominal wall fastener in FIG. 15 are attached and detached to/from each other; FIG. 17 is a perspective view showing a configuration of a coil spring in the intracorporeal attitude adjuster of the intra-abdominal camera; and FIG. 18 is a perspective view showing a configuration of a self-aligning bearing in the intracorporeal attitude adjuster of the intra-abdominal camera.

As shown in FIG. 1, the endoscope system 1 according to the present embodiment used for laparoscopic surgery mainly includes a rigid endoscope 2 which is a first photographic apparatus, an extracorporeal apparatus 3 which is an extracorporeal attitude control apparatus, a very small intra-abdominal camera (hereinafter simply referred to as a camera) 4 which combines a second photographic apparatus and image pickup apparatus, a light source 5, a camera control unit (hereinafter abbreviated to CCU) 6 which is a signal processing unit with a built-in image processing circuit, a display apparatus 7 which, being connected to the CCU 6 via a communications cable 13, displays observation images.

The light source 5 supplies illuminating light to an illumination optical system of the rigid endoscope 2. The light source 5 and rigid endoscope 2 are detachably interconnected by a light source cable 10.

The rigid endoscope 2 mainly includes an insertion portion 8 which is rigid and an operation portion 9 linked to a proximal end portion of the insertion portion 8. An image guide and light guide bundle are passed through the insertion portion 8 of the rigid endoscope 2. Also, a photographic optical system and the illumination optical system are disposed on a distal end face of the insertion portion 8, where the photographic optical system focuses a subject image on a camera (described later) for the rigid endoscope via the image guide and the illumination optical system directs the illuminating light from the light guide bundle at a subject.

The operation portion 9 of the rigid endoscope 2 incorporates a camera head (not shown) which contains a solid image pickup device such as a CCD or CMOS. An optical image of an observed site illuminated by the illuminating light supplied to the rigid endoscope 2 from the light source 5 via the light source cable 10 is picked up by the camera head in the operation portion 9 via the image guide of the insertion portion 8. The camera for the rigid endoscope photoelectrically converts the picked-up optical image into an image pickup signal, which is transmitted to the CCU 6 via an image pickup cable 11. An image pickup optical system of the rigid endoscope 2 according to the present embodiment is configured such that an angle of view a (see FIG. 8) available for photographing will be 70° to 75°.

The CCU 6 generates a video signal from the transmitted image signal and outputs the video signal to the display apparatus 7. The display apparatus 7 is, for example, a liquid crystal display. The display apparatus 7 receives the video signal outputted from the CCU 6, and displays a normal observation image from the rigid endoscope 2 and wide-angle observation image from the camera 4 on a two-part split screen or displays the two types of observation image separately by switching between the two types. The CCU 6 is detachably connected with the extracorporeal apparatus 3 via an electrical cable 12.

Next, the extracorporeal apparatus 3 will be described in detail below with reference to FIGS. 2 and 3.

As shown in FIGS. 2 and 3, the extracorporeal apparatus 3 has a receiver 31 in a casing 21. An extracorporeal attitude adjuster 22 which is an extracorporeal attitude controller is rotatably installed in the casing 21 made of non-magnetic material, where the extracorporeal attitude adjuster 22 has a spherical shape part of which has been severed.

The extracorporeal attitude adjuster 22 has a spherical body made of non-magnetic material, i.e., a synthetic resin such as plastics, with a hole 23 passing through the spherical body at the center and with part (lower part, in this case) of the spherical body severed to form a flat portion 24. The extracorporeal attitude adjuster 22 has an extracorporeal permanent magnet 25 disposed around the hole 23, the extracorporeal permanent magnet 25 being a cylindrical extracorporeal ferromagnetic member. The extracorporeal permanent magnet 25 has N and S poles separated by a plane along the hole 23.

The extracorporeal attitude adjuster 22 is movably placed in a spherical recess 26 which has a similar spherical shape and opens at top of the casing 21. The extracorporeal apparatus 3 has a so-called trackball mechanism which makes the extracorporeal attitude adjuster 22 rotatable with respect to the casing 21.

The casing 21 has a wire passage hole 27 which is communicated with the spherical recess 26 at a bottom center of the spherical recess 26 and is located on an extension of a center line of the extracorporeal attitude adjuster 22 in such a way as to open to an underside of the casing 21. Also, in the casing 21, a wire fastening lever 32 (described later) is slidably formed in a lateral direction, being communicated with the wire passage hole 27 and a slide hole 28 is formed, opening up on one side (on right side, in this case). Furthermore, a screw hole 29 is formed in the casing 21, being communicated with the spherical recess 26 and opening up on the other side (on left side, in this case), where an attitude locking screw 35 (described later) is screwed into the screw hole 29.

Being made of non-magnetic material, the wire fastening lever 32 with a bias spring 34 fastened to an end face is inserted in the slide hole 28 in the casing 21. The wire fastening lever 32, which is approximately rectangular in shape, has a hole 33 which is communicated with the wire passage hole 27 in the casing 21 as the wire fastening lever 32 slides inward in the casing 21.

The attitude locking screw 35 is made of non-magnetic material and screwed into the screw hole 29 of the casing 21 to serve as an attitude lock. When the attitude locking screw 35 is screwed deeply enough into the screw hole 29, the extracorporeal attitude adjuster 22 abuts an inner edge face of the casing 21, restraining movement of the extracorporeal attitude adjuster 22 in the spherical recess 26.

Next, the camera 4 will be described in detail below with reference to FIGS. 4 and 5.

As shown in FIGS. 4 and 5, the camera 4 mainly includes a camera body 41 and abdominal wall fastener 42 which are coupled in a row.

The camera body 41 includes a so-called capsule-type image pickup unit 43 and an intracorporeal attitude adjuster 44 which is a driven attitude controller.

At a distal end (on lower side in FIG. 4), exterior of the image pickup unit 43 is formed by a transparent hood 51 shaped like a dome and a camera casing 52 made of non-magnetic material and configured such that one face will be hermetically sealed by the transparent hood 51.

On one face of the camera casing 52 on the side of the transparent hood 51, there are a plurality of (two in this case) white LEDs 53 which are illuminators serving as a light source of illuminating light. Also, the camera casing 52 contains an objective lens group 54 held in a lens holding hole formed in an approximate center of the one face as well as a solid image pickup unit 55 such as a CCD or CMOS whose light-receiving unit is disposed at a location on which photographic light is focused by the objective lens group 54.

Also, the camera casing 52 contains a transmitter 57 and a battery 56. The battery 56 supplies power to the transmitter 57, white LEDs 53, and solid image pickup unit 55. A functional portion of the camera body 41 according to the present embodiment includes an image pickup optical system which provides such a wide coverage that an angle of view P (see FIG. 8) available for photographing will be 90° or more. An image signal produced by the solid image pickup unit 55 through photoelectric conversion is transmitted by radio from the transmitter 57 to the receiver 31 of the extracorporeal apparatus 3.

The intracorporeal attitude adjuster 44 includes a main body 61 which is made of non-magnetic material, approximately cylindrical in outer shape, and fitted into a proximal end (upper end in FIG. 4) of the camera casing 52; a spherical body 62 formed integrally with an extending end of a neck 62a which, being made of the same material as the main body 61, extends from a center of the proximal end face of the main body 61; and a spherical-body support 64 which, being made of non-magnetic material, rotatably supports the spherical body 62.

The main body 61 contains an intracorporeal permanent magnet 63 which is a cylindrical, intracorporeal ferromagnetic member. The intracorporeal permanent magnet 63 has N and S poles separated by a plane along the center of the main body 61 as shown in FIG. 5.

The spherical-body support 64 has a recess 65 to house and rotatably hold the spherical body 62. This provides a ball joint 66 in which the spherical body 62 is rotatably held in the spherical-body support 64.

An engaging connector 67 flanged outward is formed at a top center, i.e., on the side opposite from the recess 65 of the spherical-body support 64. A wire retainer housing recess 68 is formed at a surface center (top face in FIG. 4) of the engaging connector 67.

The abdominal wall fastener 42, which is made of flexible, elastic material, includes a connector 71 detachably connected with the engaging connector 67 of the spherical-body support 64 and a suction cup 72 located at a rear end of the connector 71.

The connector 71 of the abdominal wall fastener 42 includes an engaging recess 71a formed at a forward-end center, i.e., on the opposite side from the suction cup 72; a protrusion 73 which protrudes cylindrically from an approximate surface center of the suction cup 72; and a wire passage hole 74 formed to be open at the protrusion 73 and engaging recess 71 a.

A wire 45 for suspension with a predetermined length is passed through the wire passage hole 74 of the abdominal wall fastener 42. One end of the wire 45 is formed into a loop. The loop portion of the wire 45 is passed through a plate-type wire retainer 46 to be fastened.

The wire retainer 46 catches on a bottom face of the engaging recess 71a of the suction cup 72 to prevent the wire 45 from coming off the suction cup 72. That is, the wire 45 is installed extending from a center of the suction cup 72. The wire retainer 46 is housed in the wire retainer housing recess 68 of the spherical-body support 64 when the abdominal wall fastener 42 is coupled with the spherical-body support 64.

That is, as shown in FIGS. 6 and 7, the abdominal wall fastener 42 is detachably attached to the spherical-body support 64 of the ball joint 66 in the camera body 41. Specifically, since the abdominal wall fastener 42 is made of a flexible, elastic material such as silicon rubber, the connector 71 is deformable, allowing the engaging connector 67 to be attached and detached to/from the engaging recess 71 a.

The engaging connector 67, which is flanged outward, is restrained in the engaging recess 71 a, making the abdominal wall fastener 42 less liable to come off the spherical-body support 64, but the present invention is not limited thereto, and may adopt a screw configuration to make the abdominal wall fastener 42 and spherical-body support 64 attachable and detachable to/from each other.

The endoscope system 1 according to the present embodiment with the above configuration is used for laparoscopic surgery and treatment of an abdominal cavity which is one of the body cavities of a patient as shown in FIG. 8.

As shown in FIGS. 9 and 10, after the rigid endoscope 2 is inserted into an abdominal cavity 101 through one trocar 110, a treatment instrument 120 such as grasping forceps is inserted into the abdominal cavity 101 through another trocar 111, and the extracorporeal apparatus 3 is placed on the abdomen of a patient 100, the endoscope system 1 according to the present embodiment is used with the extracorporeal apparatus 3 and camera 4 sandwiching an abdominal wall 102.

Incidentally, the camera 4 is inserted into the abdominal cavity 101 through the other trocar 111. A puncture needle or other similar treatment instrument (not shown) which is passed through the extracorporeal apparatus 3 sticks into the abdominal cavity 101 from outside the body, hooks up the wire 45 of the camera 4 introduced into the abdominal cavity 101, and draws the wire 45 out of the body of the patient 100 so that the wire 45 will pass through the extracorporeal apparatus 3 as shown in FIG. 10.

When the camera 4 is introduced into the abdominal cavity 101 through the trocar 111, the protrusion 73 which protrudes cylindrically from an approximate surface center of the suction cup 72 is grasped by a treatment instrument such as grasping forceps. The protrusion 73, which is provided in the approximate center of the adhering surface of the suction cup 72, allows the camera 4 to be grasped in a balanced manner. Consequently, when introducing the camera 4 into an abdominal cavity, a surgeon can pass the camera 4 easily through the trocar 111, i.e., introduce the camera 4 easily into the abdominal cavity 101 without causing the camera 4 to get caught in the trocar 111.

When it is confirmed, based on images from the rigid endoscope 2, that the suction cup 72 of the camera 4 is placed in intimate contact with an inner surface of the abdominal wall 102, the surgeon stops pushing in the wire fastening lever 32 of the extracorporeal apparatus 3. This causes the wire fastening lever 32 of the extracorporeal apparatus 3 to move under a biasing force of the bias spring 34 as shown in FIG. 10, throwing the hole 33 out of alignment with the wire passage hole 27 in the casing 21. Consequently, the wire 45 passing through the hole 33 and the wire passage hole 27 is pinched and fastened to the casing 21, causing the extracorporeal apparatus 3 and camera 4 to be fastened with the abdominal wall 102 sandwiched between them.

In this way, with the camera 4 placed stably in the abdominal cavity 101 of the patient 100, the surgeon performs laparoscopic surgery using the endoscope system 1 according to the present embodiment. Incidentally, with one end of an insufflation tube (not shown) attached, for example, to the trocar 110, carbon dioxide gas, for example, is injected into the body cavity as an insufflation gas to ensure field of view of the rigid endoscope 2 as well as to provide an area for manipulation of surgical instruments. Then, with the camera 4 kept in intimate contact with the abdominal wall 102 in the abdominal cavity 101, the surgeon performs laparoscopic surgery by inserting the rigid endoscope 2 and treatment instrument 120 through the trocar 110 and trocar 111, respectively.

Now, operation of the extracorporeal apparatus 3 and camera 4 of the endoscope system 1 according to the present embodiment will be described in detail with reference to FIGS. 11 and 12.

As shown in FIG. 11, when the extracorporeal attitude adjuster 22 of the extracorporeal apparatus 3 rotates (in direction R in FIG. 11) around an axis A parallel to the hole 23 passing through the center, the intracorporeal permanent magnet 63 which is subjected to magnetic force of the extracorporeal permanent magnet 25 follows the rotation and the camera 4 rotates around a long axis a (in direction r in FIG. 11).

Specifically, the intracorporeal permanent magnet 63 is constantly subjected to the magnetic force of the extracorporeal permanent magnet 25, with the S pole of the intracorporeal permanent magnet 63 being attracted to the N pole of the extracorporeal permanent magnet 25 and the N pole of the intracorporeal permanent magnet 63 being attracted to the S pole of the extracorporeal permanent magnet 25. Consequently, following the rotation of the extracorporeal attitude adjuster 22 around the axis A, the camera body 41 of the camera 4 rotates using a center of the spherical body 62 of the ball joint 66 as a fulcrum.

This allows the surgeon to rotate the camera body 41 by manipulating the extracorporeal attitude adjuster 22 of the extracorporeal apparatus 3, and thus the surgeon can rotate an image picked up by the image pickup unit 43 for display on the display apparatus 7 and thereby adjust left/right and top/bottom display positions of the abdominal cavity. That is, by manipulating the extracorporeal attitude adjuster 22 of the extracorporeal apparatus 3, the surgeon can change left, right, top, and bottom positions of an image shot by the camera 4 according to left, right, top, and bottom positions of an image taken by the rigid endoscope 2, in a contactless manner using the magnetic force. Thus, the surgeon can match left-right and top-bottom directions between two images taken by the rigid endoscope 2 and camera 4 and thereby avoid feeling odd when watching the display apparatus 7.

As shown in FIG. 12, when the extracorporeal attitude adjuster 22 of the extracorporeal apparatus 3 is rotated a predetermined angle γ in the left-right direction (in direction R in FIG. 12), the intracorporeal permanent magnet 63 of the camera 4 is attracted and tilts a predetermined angle δ in the left-right direction (in direction r in FIG. 12) by the magnetic force of the extracorporeal permanent magnet 25. The predetermined angles y and 6 depend on mass of the camera body 41 of the camera 4, magnetic intensities of the extracorporeal permanent magnet 25 and intracorporeal permanent magnet 63, and the like.

That is, when the S pole of the extracorporeal permanent magnet 25 comes close to the intracorporeal permanent magnet 63 due to the rotation, the magnetic force of the extracorporeal permanent magnet 25 attracting the N pole of the intracorporeal permanent magnet 63 increases. However, the N pole of the extracorporeal permanent magnet 25 goes away from the intracorporeal permanent magnet 63 due to the rotation, decreasing the magnetic force of the extracorporeal permanent magnet 25 attracting the S pole of the intracorporeal permanent magnet 63. Consequently, the extracorporeal attitude adjuster 22 rotates by the predetermined angle y in the left-right direction, causing the camera body 41 of the camera 4 to tilt by the predetermined angle δ with the center of the spherical body 62 of the ball joint 66 serving as a fulcrum.

This allows the surgeon to tilt the camera body 41 in a contactless manner using the magnetic force by manipulating the extracorporeal attitude adjuster 22 of the extracorporeal apparatus 3, making it possible to adjust display position of an affected area picked up by the image pickup unit 43 for display on the display apparatus 7. For example, the surgeon can bring an image of the affected area to be treated to an approximate center of a screen or to a position where it is easy to treat the affected area. That is, the surgeon can change a shooting direction of a camera 4 in the abdominal cavity 101.

The surgeon can fix the camera 4 in a desired observation direction by screwing the attitude locking screw 35 into the casing 21 and thereby fastening the extracorporeal attitude adjuster 22.

The endoscope system 1 according to the present embodiment allows the surgeon to observe body tissue in a body cavity—the abdominal cavity 101 in this case—from multiple viewpoints including a wide angle viewpoint. For example, the surgeon can see an entire resection line during surgery of a large organ or resection of the large intestine. Also, the endoscope system 1 allows the surgeon to easily change line of sight of the camera 4 introduced into the abdominal cavity 101 apart from the rigid endoscope 2 for normal observation as well as fix the line of sight. Consequently, use of the endoscope system 1 according to the present embodiment makes it easy to administer treatment by means of laparoscopic surgery.

Major components of the extracorporeal apparatus 3, including the casing 21, the extracorporeal attitude adjuster 22, and the wire fastening lever 32, but excluding the extracorporeal permanent magnet 25, are made of non-magnetic material. Also, components of the camera 4, including the abdominal wall fastener 42 and intracorporeal attitude adjuster 44, but excluding the intracorporeal permanent magnet 63, are made of non-magnetic material. That is, the components installed between the extracorporeal permanent magnet 25 of the extracorporeal apparatus 3 and the intracorporeal permanent magnet 63 of the camera 4 are made of non-magnetic material. This is designed to prevent the camera 4 from affecting the magnetism of the extracorporeal permanent magnet 25 and the intracorporeal permanent magnet 63 used by the extracorporeal apparatus 3 for attitude adjustment operations.

The abdominal wall fastener 42, which is made of a flexible, elastic material, may be damaged, for example, when the protrusion 73 is grasped with grasping forceps or may be otherwise degraded as a result of repeated use, resulting in reduced adhesion to the abdominal wall 102. Thus, the abdominal wall fastener 42 according to the present embodiment has a separable structure which makes the abdominal wall fastener 42 replaceable by being attached and detached to/from the camera body 41. Of course, the abdominal wall fastener 42 may be designed to be disposable.

With the endoscope system 1 according to the present embodiment, in which the abdominal wall fastener 42 is separable from the camera body 41, only the abdominal wall fastener 42 which can be manufactured at relatively low costs needs to be replaced depending on degradation of the abdominal wall fastener 42 such as damage to the protrusion 73 or reduced adhesion to the abdominal wall 102. This offers great economy and allows the camera 4 to be fastened to the abdominal wall 102 in a stable manner.

Furthermore, although the wire 45 from the abdominal wall fastener 42 is difficult to clean for reuse, since the abdominal wall fastener 42 is removable from the camera body 41, the wire 45 can be removed easily from the abdominal wall fastener 42, and thus be made disposable.

Also, since the camera 4 in the endoscope system 1 has the abdominal wall fastener 42 equipped with the suction cup 72 which is left in intimate contact with soft body cavity tissue, i.e., the abdominal wall 102 in this case, the camera 4 can be left fastened to the abdominal wall 102 in a stable manner. Also, in the endoscope system 1 according to the present embodiment, since the camera 4 is left fastened to the abdominal wall 102 in a stable manner, the line of sight of the camera 4 can be changed in a stable manner using the extracorporeal apparatus 3.

Incidentally, although it has been stated that the abdominal wall fastener 42 is removable from the camera body 41 of the camera 4 in the endoscope system 1, this is not restrictive, and configurations shown in FIGS. 13 to 16 may be used as well.

Specifically, as shown in FIGS. 13 and 14, the ball joint 66 and the main body 61 on the side of the image pickup unit 43 may be configured to be attachable and detachable to/from each other by cutting external threads 62b on the neck 62a of the spherical body 62 and forming a female screw 61 a which screws over the external threads 62b in a center of that end face of the main body 61 which is located on the side of the spherical body 62. Also, the ball joint 66 which is difficult to clean may be configured to be disposable.

Also, as shown in FIGS. 15 and 16, the image pickup unit 43 and intracorporeal attitude adjuster 44 may be configured to be attachable and detachable to/from each other by forming a female screw 61b in a center of that end face of the main body 61 which is located on the side of the image pickup unit 43 and forming a male screw 52a which screws into the female screw 61b, at a center of an end face of the camera casing 52 of the image pickup unit 43.

As shown in FIG. 17, in the intracorporeal attitude adjuster 44 of the camera 4, a coil spring 81 may be installed between the main body 61 and abdominal wall fastener 42 instead of the ball joint 66 so that the attitude of the image pickup unit 43 can be adjusted by the extracorporeal apparatus 3. The coil spring 81 is configured to be attachable and detachable to/from the abdominal wall fastener 42 in a manner similar to the outwardly flanged engaging connector 67.

Furthermore, the intracorporeal attitude adjuster 44 of the camera 4 may be configured such that the attitude of the image pickup unit 43 can be adjusted using a so-called self-aligning bearing mechanism 85 such as shown in FIG. 18 by the extracorporeal apparatus 3.

For example, even if some of the components of the embodiment are removed, as long as the problems to be solved by the invention can be solved and the advantages of the invention are available, the resulting configuration can constitute an invention.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A medical apparatus comprising:
a medical device equipped with a driven attitude controller and introduced into a body cavity;
a fastener detachably installed on the medical device and used for fastening to a body wall in the body cavity; and
an attitude control apparatus equipped with an attitude controller which moves the medical device with respect to the fastener.

2. The medical apparatus according to claim 1, further comprising a movable unit which, being interposed between the medical device and the fastener, movably connects the medical device to the fastener.

3. The medical apparatus according to claim 1, wherein the fastener comprises a suction cup which, being made of a flexible material, is used for fastening by adhering closely to the body wall.

4. The medical apparatus according to claim 2, wherein the fastener comprises a suction cup which, being made of a flexible material, is used for fastening by adhering closely to the body wall.

5. The medical apparatus according to claim 1, wherein the attitude control apparatus moves the medical device from outside a body.

6. The medical apparatus according to claim 2, wherein the attitude control apparatus moves the medical device from outside a body.

7. The medical apparatus according to claim 3, wherein the attitude control apparatus moves the medical device from outside a body.

8. The medical apparatus according to claim 4, wherein the attitude control apparatus moves the medical device from outside a body.

9. The medical apparatus according to claim 1, wherein the fastener can be attached and detached to/from the driven attitude controller.

10. The medical apparatus according to claim 2, wherein the fastener can be attached and detached to/from the driven attitude controller.

11. The medical apparatus according to claim 3, wherein the fastener can be attached and detached to/from the driven attitude controller.

12. The medical apparatus according to claim 4, wherein the fastener can be attached and detached to/from the driven attitude controller.

13. The medical apparatus according to claim 5, wherein the fastener can be attached and detached to/from the driven attitude controller.

14. The medical apparatus according to claim 6, wherein the fastener can be attached and detached to/from the driven attitude controller.

15. The medical apparatus according to claim 7, wherein the fastener can be attached and detached to/from the driven attitude controller.

16. The medical apparatus according to claim 8, wherein the fastener can be attached and detached to/from the driven attitude controller.
